(19) United States
(12) Patent Application Publication
(10) Pub. No.: US 2025/0120592 A1
(43) Pub. Date: Apr. 17, 2025

(54) SYSTEMS AND METHODS FOR DETECTING BIOMETRIC AND NEUROPHYSIOLOGICAL PARAMETERS

(71) Applicant: NIRSense, Inc., Richmond, VA (US)

(72) Inventors: Ryan Casey BOUTWELL, Richmond, VA (US); Thibault Pierre Thierry ROUMENGOUS DE FESTES, Richmond, VA (US); Jason Michael STROHMAIER, Richmond, VA (US); Bryan Oliver HELLRIEGEL, Richmond, VA (US); Jared Dale ALLEN, Richmond, VA (US); Daniel Collins Rodenhaver, Richmond, VA (US)

(73) Assignee: NIRSense, Inc., Richmond, VA (US)

(21) Appl. No.: 18/987,712

(22) Filed: Dec. 19, 2024

Related U.S. Application Data
(63) Continuation-in-part of application No. PCT/US2023/069227, filed on Jun. 28, 2023.
(60) Provisional application No. 63/356,740, filed on Jun. 29, 2022.

(51) Int. Cl. A61B 5/0205; A61B 5/00; A61B 5/01; A61B 5/021; A61B 5/024; A61B 5/053; A61B 5/08; A61B 5/1455; A61B 5/291; A61B 5/296; A61B 5/297

(52) U.S. Cl. CPC A61B 5/0205; A61B 5/6804; A61B 5/6887; A61B 5/7271; A61B 5/01; A61B 5/02141; A61B 5/02427; A61B 5/053; A61B 5/0803; A61B 5/0816; A61B 5/14552; A61B 5/291; A61B 5/296; A61B 5/297; A61B 2560/045; A61B 2560/0462; A61B 2562/0233; A61B 2562/164

(57) ABSTRACT

A system may include a substrate in contact with a user. The substrate may include first detector(s) capable of detecting optical biometric properties of the user, and second detector(s) capable of detecting non-optical biometric properties of the user. The system may include a first and a second electronics module communicatively coupled to the substrate. Each of the two electronics modules may include a processor, a memory device, and instructions stored on the memory device that, when executed, direct the processor to perform step(s). These step(s) may include receiving a signal from the first detector(s) or second detector(s), processing the signal based on predefined algorithm(s), and determining, based on the processed signal, whether the user requires one or more feedback actions.

### CROSS-REFERENCE TO RELATED APPLICATIONS

This application is a continuation-in-part (CIP) application of, and claims priority under 35 U.S.C. § 111 (a) to, International Application No. PCT/US23/69227, filed Jun. 28, 2023, which claims priority under 35 U.S.C. § 119 to U.S. Provisional Patent Application No. 63/356,740, filed Jun. 29, 2022, the entire contents of each of which are fully incorporated herein by reference.

### TECHNICAL FIELD

The present disclosure relates generally to systems and methods for detecting biometric and neurophysiological parameters. In particular, the present disclosure relates to systems and methods for processing optical and non-optical properties of a user.

### BACKGROUND

Near-infrared spectroscopy (NIRS) devices interrogate biological tissue using a selection of light wavelengths in the red and near-infrared (NIR) region of the electromagnetic spectrum. These wavelengths are particularly well suited for deep light penetration through tissue, versus lower wavelengths of light that are scattered or absorbed by confounding factors in the body and thus cannot reach the tissue depth of these red and NIR wavelengths. NIRS devices generally feature at least two wavelengths of light output in this range and at least one detector, and including additional optical elements can allow different depths of sensing.

Red and near-infrared wavelengths are particularly effective for non-invasively sensing different molecular states of hemoglobin in various body tissues. Unfortunately, existing NIRS devices are typically expensive, large desktop units with disintegrated sensor and processing systems. This lack of portability limits the usefulness of NIRS outside of the surgical suite, laboratory, and research environments. Some portable solutions include a sensor-only patch with wired communication to a separate portable, pocketable, or head-worn processing and communications unit. These changes represent only a nominal improvement, as the processing unit is itself not fully wearable and risks physically detaching from the sensor unit through movement or cable weight. These limitations greatly decrease the wearability and utility of such systems. These semi-ambulatory systems are also typically not designed to be used in parallel, where individual NIRS sensor systems work in tandem across the body or across a population to continually sense physiological features at multiple places using a common interface. Non-ambulatory systems can have more sensor inputs, but these are limited by the total number of ports designed into the physical system itself. Therefore, there exists a need for integrated NIRS systems and methods of using those systems to interrogate biological tissue.

Additionally, existing NIRS systems and methods are unable to be coupled with electrophysiological sensing capabilities in an integrated and/or wearable package. Most portable, wearable electrophysiological sensing systems, such as those used in electroencephalography (EEG), use a very small number of electrodes for cognitive sensing and do not have the additional benefit of optical hemodynamic sensing for additional contextual awareness.

### SUMMARY

In some embodiments, a system may include a substrate in contact with a user. The substrate may include one or more first detectors capable of detecting one or more optical biometric properties of the user, and one or more second detectors capable of detecting one or more non-optical biometric properties of the user. The system may further include a first electronics module communicatively coupled to the substrate. The first electronics module may include a first processor, a first memory device, and first instructions stored on the first memory device that, when executed, direct the first processor to perform one or more steps. These one or more steps may include receiving a first signal from the one or more first detectors, processing the first signal based on one or more first predefined algorithms, and determining, based on the processed first signal, whether the user requires one or more first feedback actions. The system may further include a second electronics module communicatively coupled to the substrate. The second electronics module may include a second processor, a second memory device, and second instructions stored on the second memory device that, when executed, direct the second processor to perform one or more steps. These one or more steps may include receiving a second signal from the one or more second detectors, processing the second signal based on one or more second predefined algorithms, and determining, based on the processed second signal, whether the user requires one or more second feedback actions. At least one of the first processor and the second processor may, responsive to at least one of the determining instructions, determine that the user requires no feedback actions, initiate at least one of the one or more first feedback actions, or initiate at least one of the one or more second feedback actions.

In some embodiments, the substrate may include a first light source capable of emitting a first set of wavelengths of red or near-infrared light. The one or more first detectors may be capable of detecting the first set of wavelengths and may be mounted on the substrate at a first distance from the first light source. The first instructions may further direct the first processor to detect a physical configuration of the substrate, select, based on the physical configuration, the first set of wavelengths, and select, based on the physical configuration, the first distance from the first light source, wherein receiving the first signal may be based on the physical configuration.

In some embodiments, the one or more optical biometric properties may include one or more of tissue oxygenation, heart rate, respiratory rate, oxygen saturation, blood pressure, or combinations thereof.

In some embodiments, the one or more non-optical biometric properties may include one or more of EEG, electrooculography (EOG), electromyography (EMG), bioimpedance circuitry, thermal properties, mechanical properties, or combinations thereof.

In some embodiments, the one or more first and second feedback actions may include one or more of an optical biometric property reading, a non-optical biometric property reading, one or more types of stimulation, or combinations thereof.

In some embodiments, the substrate may include one or more terminals capable of providing a user with the one or more types of stimulation, which may include one or more of transcranial direct current stimulation (tDCS), transcranial alternating current stimulation (tACS), vagus nerve stimulation (nVNS), transcutaneous electrical nerve stimulation (TENS), transcranial magnetic stimulation (TMS), ultrasound stimulation, optical stimulation, mechanical stimulation, or combinations thereof.

In some embodiments, the one or more first and second feedback actions may be provided to the user by changes in a real or virtual environment of the user, and/or by pharmacological changes.

In some embodiments, the one or more first and second feedback actions may be perceived by the user, may not be perceived by the user, or may be partially perceived by the user.

In some embodiments, the one or more first and second feedback actions may be provided to a user via an environmental property. The environmental property may include one or more of temperature, pressure, chemical composition, sound, light, motion, or combinations thereof.

In some embodiments, the system may include a flexible material coupled to the substrate to improve contact with the user. In some embodiments, the flexible material and the substrate may be configured to removably contact one or more of a body part of the user, an article of clothing, a piece of equipment, a prosthetic, or combinations thereof.

In some embodiments, a system may include a substrate in contact with a user. The substrate may include one or more first detectors capable of detecting one or more optical biometric properties of the user, one or more second detectors capable of detecting one or more non-optical biometric properties of the user, and one or more terminals capable of providing the user with one or more types of stimulation. The system may further include a first electronics module communicatively coupled to the substrate and including a first processor, a first memory device, and first instructions stored on the first memory device that, when executed, may direct the first processor to perform one or more steps. The one or more steps may include receiving a first signal from the one or more first detectors, processing the first signal based on one or more first predefined algorithms, and determining, based on the processed first signal, whether the user requires one or more first types of stimulation. The system may further include a second electronics module communicatively coupled to the substrate and including a second processor, a second memory device, and second instructions stored on the second memory device that, when executed, direct the second processor to perform one or more steps. The one or more steps may include receiving a second signal from the one or more second detectors, processing the second signal based on one or more second predefined algorithms, and determining, based on the processed second signal, whether the user requires one or more second types of stimulation. At least one of the first processor and the second processor may, responsive to at least one of the determining instructions, determine that the user requires no types of stimulation, initiate, via the one or more terminals, at least one of the one or more first types of stimulation, or initiate, via the one or more terminals, at least one of the one or more second types of stimulation.

In some embodiments, the substrate may include a first light source capable of emitting a first set of wavelengths of red or near-infrared light. The one or more first detectors may be capable of detecting the first set of wavelengths and may be mounted on the substrate at a first distance from the first light source. The first instructions may further direct the first processor to detect a physical configuration of the substrate, select, based on the physical configuration, the first set of wavelengths, and select, based on the physical configuration, the first distance from the first light source, wherein receiving the first signal is based on the physical configuration.

In some embodiments, the one or more optical biometric properties may include one or more of tissue oxygenation, heart rate, respiratory rate, oxygen saturation, blood pressure, or combinations thereof.

In some embodiments, the one or more non-optical biometric properties may include one or more of EEG, EOG, EMG, bioimpedance circuitry, thermal properties, mechanical properties, or combinations thereof.

In some embodiments, the one or more first and second types of stimulation may include one or more of tDCS, tACS, nVNS, TENS, TMS, ultrasound stimulation, optical stimulation, mechanical stimulation, or combinations thereof.

In some embodiments, the system may include a flexible material coupled to the substrate to improve contact with the user. In some embodiments, the flexible material and the substrate may be configured to removably contact one or more of a body part of the user, an article of clothing, a piece of equipment, a prosthetic, or combinations thereof.

In some embodiments, a system may include a substrate in contact with a user. The substrate may include one or more first detectors capable of detecting one or more optical biometric properties of the user, and one or more second detectors capable of detecting one or more non-optical biometric properties of the user. The system may further include an electronics module communicatively coupled to the substrate. The electronics module may include one or more processors, one or more memory devices, and one or more instructions stored on the one or more memory devices that, when executed, direct the one or more processors to perform one or more steps. The one or more steps may include receiving a first signal from the one or more first detectors, receiving a second signal from the one or more second detectors, processing the first signal based on one or more first predefined algorithms, processing the second signal based on one or more second predefined algorithms, determining, based on the processed first signal, whether the user requires one or more first feedback actions, and determining, based on the processed second signal, whether the user requires one or more second feedback actions. At least one of the one or more processors may, responsive to at least one of the determining instructions, determine that the user requires no feedback actions, initiate at least one of the one or more first feedback actions, or initiate at least one of the one or more second feedback actions.

In some embodiments, the substrate may include one or more terminals capable of providing a user with the one or more types of stimulation, which may include one or more of tDCS, tACS, nVNS, TENS, TMS, ultrasound stimulation, optical stimulation, mechanical stimulation, or combinations thereof.

In some embodiments, a method of calculating one or more biometric properties may include mounting a system as disclosed herein on a user, executing the first instructions for a first period of time to determine a first baseline level associated with the one or more optical biometric properties, regularly executing the first instructions to process the first signal based on the one or more first predefined algorithms, determining, based on the processed first signal, whether a user requires one or more first feedback actions, executing the second instructions for a second period of time to determine a second baseline level associated with the one or more non-optical biometric properties, regularly executing the second instructions to process the second signal based on the one or more second predefined algorithms, and determining, based on the processed second signal, whether the user requires one or more second feedback actions. At least one of the first processor and the second processor may, responsive to at least one of the determining instructions, determine that the user requires no feedback actions, initiate at least one of the one or more first feedback actions, or initiate at least one of the one or more second feedback actions.

### BRIEF DESCRIPTION OF THE DRAWINGS

The accompanying drawings, which are incorporated in and form a part of the specification, illustrate the embodiments of the invention and together with the written description serve to explain the principles, characteristics, and features of the invention. In the drawings:

FIG. 1A depicts a top view of an embodiment of a substrate of a system for detecting biometric and neurophysiological parameters, in accordance with the present disclosure.

FIG. 1B depicts a side view of the embodiment of FIG. 1A of the substrate of a system for detecting biometric and neurophysiological parameters, in accordance with the present disclosure.

FIG. 1C depicts a bottom view of the embodiment of the substrate of a system for detecting biometric and neurophysiological parameters, in accordance with the present disclosure.

FIG. 1D depicts an embodiment of the substrate of a system for detecting biometric and neurophysiological parameters, in accordance with the present disclosure.

FIG. 2 depicts an embodiment of a substrate of a system for detecting biometric and neurophysiological parameters, in accordance with the present disclosure.

FIG. 3 depicts an embodiment of an electronics module of a system for detecting biometric and neurophysiological parameters, in accordance with the present disclosure.

FIG. 4 depicts an embodiment of a system for detecting biometric and neurophysiological parameters, in accordance with the present disclosure.

FIG. 5 is a flowchart of a method for detecting biometric and neurophysiological parameters, in accordance with the present disclosure.

FIG. 6 depicts an embodiment of a system for detecting biometric and neurophysiological parameters, in accordance with the present disclosure.

### DETAILED DESCRIPTION

This disclosure is not limited to the particular systems, devices, and methods described, as these may vary. The terminology used in the description is for the purpose of describing the particular versions or embodiments only, and is not intended to limit the scope of the disclosure.

The following terms shall have, for the purposes of this application, the respective meanings set forth below. Unless otherwise defined, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

As used herein, the singular forms “a,” “an,” and “the” include plural references, unless the context clearly dictates otherwise. Thus, for example, reference to a “fiber” is a reference to one or more fibers and equivalents thereof known to those skilled in the art, and so forth.

As used herein, the term “about” means plus or minus 10% of the numerical value of the number with which it is being used. For example, about 50 mm means in the range of 45 mm to 55 mm.

As used herein, the term “consists of” or “consisting of” means that the device or method includes only the elements, steps, or ingredients specifically recited in the particular claimed embodiment or claim.

In embodiments or claims where the term comprising is used as the transition phrase, such embodiments can also be envisioned with replacement of the term “comprising” with the terms “consisting of” or “consisting essentially of.”

It is to be understood that the mention of one or more method steps does not preclude the presence of additional method steps or intervening method steps between those steps expressly identified. It is also to be understood that the listing of one or more method steps in any order does not preclude the performance of such one or more methods steps in alternative orders, nor does it preclude the simultaneous performance of any such one or more method steps. Similarly, it is also to be understood that the mention of one or more components in a device or system does not preclude the presence of additional components or intervening components between those components expressly identified.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

In addition, even if a specific number is explicitly recited, those skilled in the art will recognize that such recitation should be interpreted to mean at least the recited number (for example, the bare recitation of “two components,” without other modifiers, means at least two components, or two or more components). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

Furthermore, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 components refers to groups having 1, 2, or 3 components. Similarly, a group having 1-5 components refers to groups having 1, 2, 3, 4, or 5 components, and so forth.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

Near-infrared spectroscopy devices interrogate biological tissue using a selection of light wavelengths in the red and NIR region of the electromagnetic spectrum. These wavelengths are particularly well suited for deep light penetration through tissue, versus lower wavelengths of light that are scattered or absorbed by confounding factors in the body and thus cannot reach the tissue depth of these red and NIR wavelengths. NIRS devices generally feature at minimum two wavelengths of light output in this range and at least one detector, and including additional optical elements can allow different depths of sensing.

Red and near-infrared wavelengths are particularly effective for non-invasively sensing different molecular states of hemoglobin in various body tissues. Hemoglobin is a strong absorber of light in the middle of the visible light spectrum but has a low optical extinction coefficient within the higher wavelengths of the visible range. Within the NIR wavelengths, for hemoglobin's oxygenation states, deoxy- and oxyhemoglobin's absorption spectra cross at an isosbestic point near 805 nm, allowing NIRS systems to differentiate oxygenation states of hemoglobin using light sources above and below this wavelength. With this differentiation, NIRS can be used for a variety of sensing mechanisms related to the body's circulatory and other functional systems.

Hemoglobin also allows for binding of ligands other than oxygen. These other molecular states of hemoglobin, such as carboxyhemoglobin and methemoglobin, have unique optical absorption characteristics in the NIR range. Investigating these molecular states can elucidate competitive binding and indicate histologic changes in tissue oxygenation such as tissue poisoning. Hemoglobin has a competitive binding efficiency for many molecules, such as carbon monoxide (CO), cyanide (CN-), sulfur monoxide (SO), sulfide (S2-), and others in these groups. Nitric oxide (NO) also binds to hemoglobin and can be detected optically. Investigating the NIR spectra of these additional bound states of hemoglobin can indicate tissue status and toxicity by inhibiting oxygen binding as well as enable sophisticated physiological monitoring of body systems.

NIRS systems may calculate oxygenation levels using the modified Beer-Lambert law (mBLL), which only requires one bank of light sources. Using the mBLL offers the translation of raw optical signals into actionable oxygenation details. Alternatively, NIRS systems may employ spatially resolved spectroscopy (SRS), which can use both short- and long-distance measurements. Separately, short channel information can be subtracted from long channel information to more accurately isolate, for example, brain activity and the contributions from internal (e.g., cerebral) vasculature and external (e.g., skin) vasculature.

Unfortunately, existing NIRS devices are typically expensive, large desktop units with disintegrated sensor and processing systems. This lack of portability limits the usefulness of NIRS outside of the surgical suite, laboratory, and research environments. Even in such controlled environments, these devices sometimes fail because they are difficult to integrate into a user's system when the planned testing involves any form of motion.

Some portable solutions include a sensor-only patch with wired communication to a separate portable, pocketable, or head-worn processing and communications unit. These changes represent only a nominal improvement, as the processing unit is itself not fully wearable and risks physically detaching the sensor unit through movement or cable weight. These limitations greatly decrease the wearability and utility of such systems. These semi-ambulatory systems are also typically not designed to be used in parallel, where individual NIRS sensor systems work in tandem across the body or across a population to continually sense physiological features at multiple places using a common interface. Non-ambulatory systems can have more sensor inputs, but these are limited by the total number of ports designed into the physical system itself. Therefore, there exists a need for integrated and/or modular NIRS systems and methods of using those systems to interrogate biological tissue.

Additionally, existing NIRS systems and methods are unable to be coupled with electrophysiological sensing capabilities in an integrated, modular, and/or wearable package. Most portable, wearable electrophysiological sensing systems, such as those used in electroencephalography (EEG), use a very small number of electrodes for cognitive sensing and do not have the additional benefit of optical hemodynamic sensing for additional contextual awareness.

Accordingly, the systems and methods disclosed herein may provide integrated, modular, and/or wearable optical and non-optical biometric sensing capabilities, such as a combination of electrophysiological, functional NIRS (fNIRS), and photoplethysmography (PPG)-based sensing, providing additional physiological metrics such as heart rate, respiratory rate, oxygen saturation (SpO₂), and blood pressure-related data streams in a single wearable system. The electrophysiological sensing capabilities described herein may provide electrophysiological sensors such as those used in EEG electrodes, e.g., covering portions of the frontal cortex of the brain; EOG electrodes, e.g., monitoring eye movement; EMG, e.g., monitoring muscle activation; and/or bioimpedance circuitry, e.g., monitoring tissue composition. In some embodiments, the systems disclosed herein may be modular such that different biometric and neurophysiological monitoring capabilities can be added or subtracted from the system to fit a user's specific requirements for system function, layout, size, etc. In some embodiments, the systems disclosed herein may be coupled with other biometric monitoring systems that may assess user aspects, such as bioimpedance, biochemical, thermal, and/or mechanical properties.

In some embodiments, the systems disclosed herein may provide electrical connections to a battery and an electronics module or modules that may process the electrophysiological signals and fNIRS signals separately or in tandem. The systems disclosed herein may be integrated into various types of equipment, uniforms, clothing, prosthetics, etc., for example, in an aircrew helmet at voids around the earcup, in other sections of the helmet interior, or at the nape of the neck just outside the helmet. The systems disclosed herein may also be worn as a standalone system, such as when a helmet is not worn, as in the above example, with the support electronics being integrated into, e.g., a skull cap or headband.

In some embodiments, the systems disclosed herein may provide a flexible, fan-shaped patch design that may conform to a portion of a user's body, such as the user's forehead, which may provide close coupling of the fNIRS optics and electrophysiological (e.g., EEG) electrodes to the skin, and may allow for more forehead movement and adaptation than a patch design covering the entire forehead. A major issue with EEG systems is the possibility of the electrodes lifting off from the body based on movement. The systems disclosed herein may include an adhesive having a large area to reduce the risk of electrode liftoff from the body in critical sensing locations. The systems disclosed herein may also use sweat-wicking materials in the adhesive itself which may ensure comfort over prolonged wear.

One issue with adhesive-based systems is ensuring that the design of the patch does not interfere with the hairline of the wearer, which can cause discomfort when the patch is removed. In instances where the user's forehead area is extremely small, the patch adhesive can be trimmed to ensure the patch does not touch the hairline without requiring the patch itself be cut.

In some embodiments, the systems disclosed herein may provide wings designed into the forehead patch that may allow the electrodes and fNIRS electronics to be set higher or lower on the forehead depending on the physical shape of the wearer's forehead. The wings themselves may be configured to maintain separation between the fNIRS electronics and EEG electrodes, which may decrease the potential for signal degradation to the EEG and EOG signals.

In some embodiments, the systems disclosed herein may provide a modular system whereby one or more components of the system (e.g., configured for monitoring or detecting certain properties of the user, and/or processing signals relating to those properties) can be integrated into a wearable device, such as a visor configured to be worn on a user's head. In some such embodiments where one or more of the above-mentioned components are integrated into a wearable device, other component(s) can be attached to other parts of the user's body such that the different components of the overall system can monitor the same and/or different properties of the user from different parts of the user's body (e.g., head, chest, etc.).

In some embodiments, the systems disclosed herein may be compatible with either commercial-off-the-shelf (COTS) wet silver/silver chloride (Ag/AgCl) electrodes or dry electrodes that may not require a gel/liquid interface to the skin for signal generation and quality. In some embodiments, the systems disclosed herein may provide an integrated fNIRS and EEG patch design having an adhesive area that augment the adhesive material on the COTS electrodes, which may reduce the risk of electrode separation from the forehead and may ensure longer wear duration.

Electrophysiological signals may be susceptible to electronic noise due to the low voltage nature of the raw signals themselves, which have to be sent over a significant physical distance before reaching the electronics module's frontend for filtering, amplification, and conversion. As such, in some embodiments, the systems disclosed herein may be designed to ensure that the electrophysiological signal lines (e.g., EEG) may be adequately protected from electronic noise through environmental factors and/or the fNIRS subsystem itself.

In some embodiments, the systems disclosed herein may be configured to have medical-grade elastomer encapsulation, which may allow for significant shielding integrated into the wearable substrate itself with the electrode lines running within the insulated/shielded material.

In some embodiments, the systems disclosed herein may provide electrode pad locations and cables to the electrodes that may also be locally shielded either within the elastomer or through use of a radio frequency (RF)-blocking, biocompatible combination adhesive. Shielding materials within the elastomer and/or adhesive can include thin copper sheets or other RF-blocking metallic or non-metallic material.

In some embodiments, the systems disclosed herein may provide a mold for the elastomer encapsulant that can be used to route the electrophysiological lines in a manner that may ensure that the pathway for the electrophysiological signals is separated as much as possible from the fNIRS subsystem to ensure local noise from that integrated patch area does not interfere with the electrophysiological signals. For example, while EOG signals may be less susceptible to interference than EEG signals, optimizing the cable routing within the mold may help to minimize degradation of the EOG signals. Similarly, for example, the routing for the prefrontal cortex groups (e.g., the measurement points at AF, F, and Fp using 10-20 EEG mapping) EEG lines can be molded away from the fNIRS subsystem so that signal interference remains low even if these areas are placed close together on the forehead.

In instances where the fNIRS electronics or optical signals may impact the electrophysiological (e.g., EEG) signals, or vice versa, the systems disclosed herein may be configured to adapt sampled data streams to toss out or vary filtering on either signal to account for the operation of the other subsystem. For example, if activation of the fNIRS optical electronics causes a noise spike in the electrophysiological readings, the milliseconds of data from the fNIRS subsystem firing sequence can be isolated, filtered, or discarded depending on the level and nature of the noise impacting the electrophysiological signal.

In some embodiments, the systems disclosed herein may be controlled by separate or integrated microcontroller(s) that may be configured to provide feedback to the user based on algorithms developed from the user's biometric property signals, such as oxygenation levels and electrical activity. In some embodiments, the systems disclosed herein may be combined with electrophysiological stimulation techniques such as tDCS, nVNS, TENS, and/or TMS, to stimulate the user based on situational need or perceived performance degradation. The systems and methods disclosed herein may thus be utilized in a variety of applications, such as flight environment, virtual reality, learning/training, teaming, gaming, human performance enhancement, rehabilitation acceleration, and others.

In some embodiments, the systems disclosed herein may provide electronics modules that can control sampling and data processing of the fNIRS, electrophysiological, and tDCS/nVNS subsystems. For example, the systems disclosed herein may be configured to communicate with an external system that can adapt its properties based on the data processed by the integrated fNIRS/electrophysiological system, and may trigger the tDCS/nVNS subsystem if certain criteria are met. Triggering of the tDCS/nVNS subsystem can be either local (e.g., on the electronics module) or remote (e.g., from the external communication system). In some embodiments, the electronics modules can be internal and/or external to a wearable device configured to house one or more components of the system, as further discussed below. For example, the electronics modules can be integrated into a wearable headset (e.g., a visor), and/or can be external to the wearable headset. In some embodiments, the electronics modules can communicate with other component(s) of the overall system via a wired and/or wireless (e.g., WiFi®) connection.

In some embodiments, the systems disclosed herein may allow details on the cognitive workload experienced by a user and identified by the patch system to be communicated with support personnel (e.g., in the flight environment) such that, for example, training and simulation activities may be adapted, and/or support personnel may be informed of any high burden scenarios experienced by a user (e.g., aircrew onboard a jet). In some embodiments, the use of NIRS-based hypoxia sensing can also alert, for example, aircrew and support personnel, of local or systemic failures in a user's breathing apparatus.

In some embodiments, the systems disclosed herein may allow for a user's cognitive workload data to be used to adapt training for the user based on the user's perception of task difficulty at a given time, such as in the virtual reality (VR) field. For example, if a performed task is either too difficult or too easy, the user may become disengaged from the task, while adequately difficult tasks may be likelier to maintain user engagement as they work or learn. By tracking workload levels over the short- or long-term, training or learning processes can be tailored to the capability or engagement level of the user, optimizing their progression.

In some embodiments, the systems disclosed herein may allow for tracking of user fatigue through perturbations of the NIRS and EEG signal that indicate reduced cognitive performance or inattention, while the EOG subsystem can identify changes such as drooping eyelids or changes in eye movement that could indicate the onset and/or depth of fatigue. Communications between this subsystem and the tDCS and/or nVNS subsystems can stimulate the user back into a state of increased alertness or, if these methods are insufficient, advise that the user cease activities until they have rested.

Reference will now be made in detail to example embodiments of the disclosed technology that are illustrated in the accompanying drawings and disclosed herein. Wherever convenient, the same reference numbers will be used throughout the drawings to refer to the same or like parts.

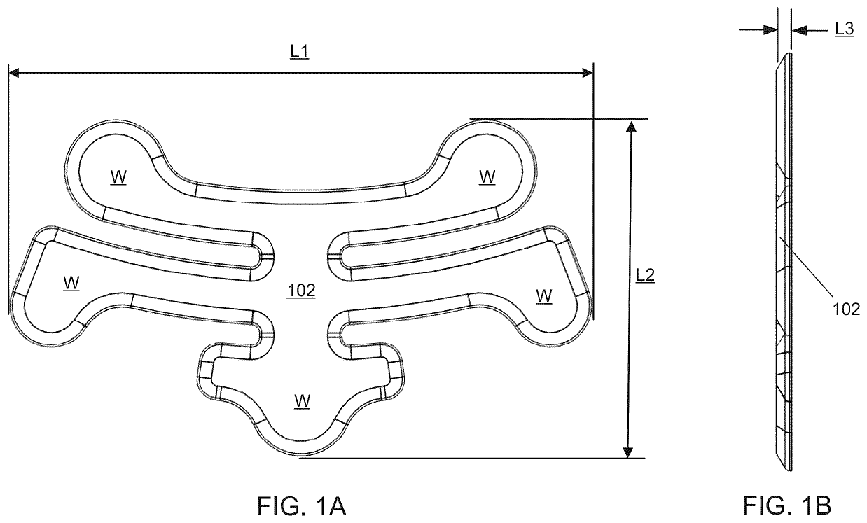

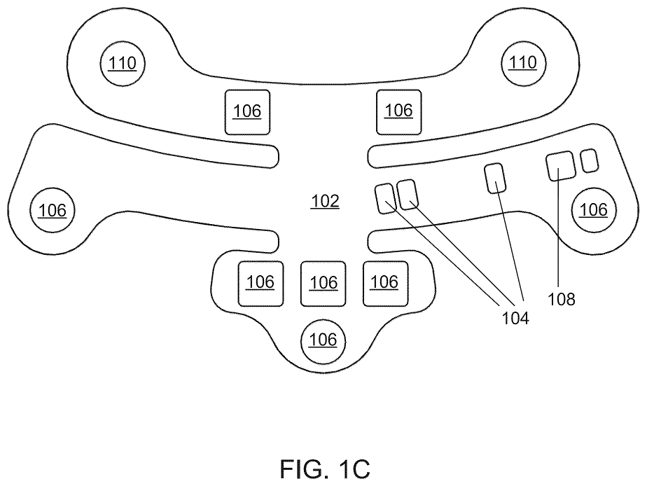

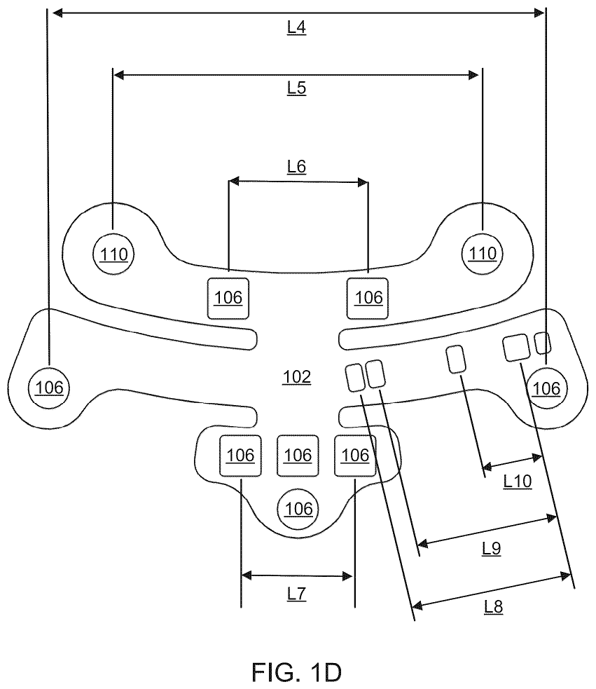

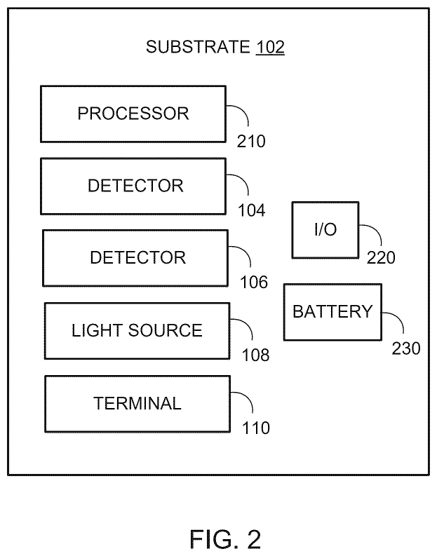

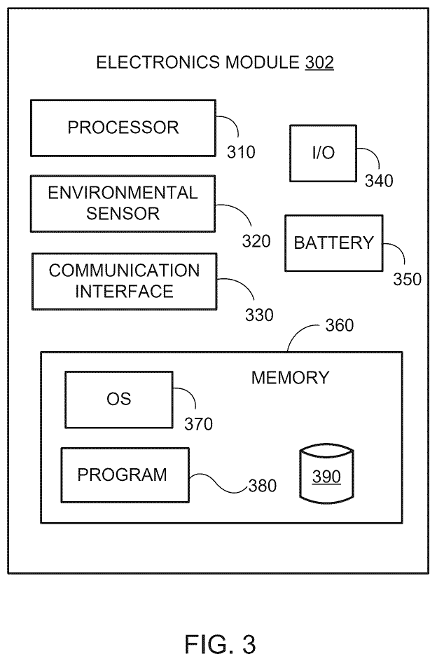

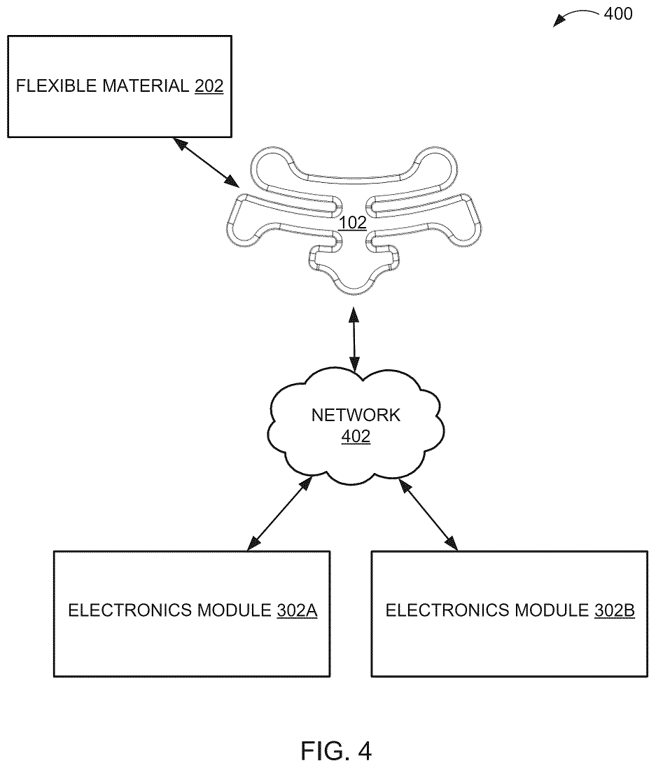

FIGS. 1A-1D and 2 depict embodiments of a substrate 102 for detecting biometric and neurophysiological parameters, in accordance with the present disclosure. The substrate 102 may be included in a system 400, along with a flexible material 202, a first electronics module (302A), and/or a second electronics module (302B), as further discussed below with respect to FIGS. 3 and 4.

In some embodiments, the substrate 102 may be coupled to a flexible material 202 (FIG. 4). For example, the substrate 102 may be configured to be placed on top of, or embedded within, a flexible material 202. As discussed herein, the flexible material 202 may improve the capability of substrate 102 to conform to a body part of a user, such as the user's forehead. The substrate 102 and/or the flexible material 202 may also provide for electrical insulation and/or optical isolation of any fNIRS and/or PPG optics, and/or any electrical connection lines to EEG electrodes mounted on substrate 102. The flexible material 202 may include one or more adhesive materials, such as sweat-resistant or sweat-wicking adhesives, such that the substrate 102 may removably adhere to a body part of the user. In some embodiments, these adhesives may provide unique optical, electrical, thermal, and/or mechanical properties that may be selected to meet sensor requirements for each area (e.g., user body part) covered by a sensor, as further discussed herein.

In some embodiments, the substrate 102 may include one or more materials, such as silicone, nylon, epoxy, a bioinert polymer, a biocompatible polymer, a woven or nonwoven textile, an adhesive film, a flexible circuit board, flexible sensors and electronics, or a combination thereof. In some embodiments, the substrate 102 and any components mounted onto the substrate may be configured to provide mechanical flexibility, allowing the substrate 102 to conform to and/or adhere to a surface. In some embodiments, the substrate 102 may be configured to be integrated into clothing or other equipment designed to be worn or applied to a user.

In some embodiments, the shape and/or dimensions of the substrate 102 may be different depending on the specific patient and/or use case. For example, as particularly shown in FIG. 1A, the substrate 102 may include a plurality of wings, W, that may allow the electrophysiological electrodes and fNIRS electronics to be set, for example, higher or lower on a user's forehead, and may provide separation between the electrophysiological electrodes and fNIRS electronics to decrease potential signal degradation between the two, as discussed herein. Additionally, substrate 102 may also be configured of any size. For example, as shown in FIG. 1A, substrate 102 may include a length L1 of approximately 5.572 inches and a width L2 of approximately 3.221 inches. As shown in FIG. 1B, substrate 102 may include a thickness L3 of approximately 0.118 inches.

As shown in FIG. 1C, in some embodiments, the substrate 102 may include one or more first detectors 104 capable of detecting one or more optical biometric properties of a user. For example, the optical biometric properties may include tissue oxygenation, heart rate, respiratory rate, oxygen saturation, blood pressure, and the like. In some embodiments, the first detectors 104 may include optical detectors configured to detect specific sets of wavelengths emitted from light source 108, as further discussed below. The optical detectors may be configured to detect backscattered light from light source 108, as the backscattered light travels through tissue. In some embodiments, the optical detectors may comprise a single optical detector. In other embodiments, the optical detectors may comprise multiple optical detectors, such as 2 optical detectors, 3 optical detectors, 4 optical detectors, 5 optical detectors, and so on. In some embodiments, the optical detectors may be capable of detecting the first set of wavelengths, as described herein. In some embodiments, the optical detectors may be capable of detecting the first set of wavelengths and the second set of wavelengths, as described herein.

In some embodiments, the substrate 102 may include one or more second detectors 106 capable of detecting one or more non-optical biometric properties of a user. For example, the non-optical biometric properties may include electrophysiological properties, such as EEG, EOG, EMG, bioimpedance circuitry, thermal properties, mechanical properties, and the like. As discussed herein, providing a substrate 102 that may detect both optical and non-optical properties of a user may enable the user to be continuously monitored in certain environments (e.g., in the flight environment). In some embodiments, as further discussed herein, this continuous monitoring may enable as-required stimulation of the user, for example, via the substrate 102 or another component of system 400 external to the substrate 102.

First detectors 104 and second detectors 106 may be mounted to substrate 102 in a variety of positions. For example, as particularly shown in FIGS. 1C-1D, first detectors 104 may be attached to one of the wings, W, of the substrate 102, and at one or more distances from a light source 108 also attached to the substrate 102. For example, first detectors 104 may be placed on the substrate 102 at distances of L8, L9, and L10 from the light source 108, where L8 may be approximately 1.575 inches, L9 may be approximately 1.378 inches, and L10 may be approximately 0.591 inches.

Second detectors 106 may be attached to one or more wings, W, of the substrate 102, and at one or more distances from one another, first detectors 104, and/or terminals 110 (further discussed below). For example, second detectors 106 may be placed on opposite wings of substrate 102 and at distances of L4 and L6 from each other, where L4 may be approximately 4.785 inches, and L6 may be approximately 1.339 inches. As another example, second detectors 106 may be placed on a single wing, W, at a distance of L7 from each other, where L7 is approximately 1.102 inches.

In some embodiments, the placement of the second detectors 106 on the substrate 102 may be based on the international 10-20 electrode system with focus on the prefrontal cortex (e.g., Fp, AF, and F groups). In some embodiments, second detectors 106 may include EEG electrodes that may be expandable from a single channel to multi-channel EEG. In some embodiments, the second detectors 106 may include EOG electrodes that may be placed around a user's eyes, for example to track eye movement. In some embodiments, the second detectors 106 may include EMG electrodes to track real-time muscle activation and/or metabolic demand.

As shown in FIG. 2, the substrate 102 may further include a processor 210, an input/output (I/O) device 220, and a power source 230 (e.g., a battery) configured to power the substrate 102.

The I/O device 220 may be configured to connect the substrate 102 to one or more other components of system 400 or one or more components external to system 400, such as a computing device (e.g., a laptop or other “smart” device).

The light source 108 may include a single light source. In other embodiments, the light source 108 may include multiple light sources, such as 2 light sources, 3 light sources, 4 light sources, 5 light sources, and so on. In some embodiments, each light source may include one or more light emitting diodes (LEDs). In some embodiments, each light source may include a single tunable light source such as a broadband LED coupled with a miniature monochromator. In some embodiments, each light source may include one or more laser diodes. In an embodiment, the light source 108 may include a light source driver capable of selecting between the different light sources or selecting the wavelength from a tunable light source.

In some embodiments, the light source 108 may be capable of emitting a first set of wavelengths of red or near-infrared light. In some embodiments, each light source within the light source 108 may be capable of independently emitting a wavelength. The first set of wavelengths may comprise 1 wavelength, 2 wavelengths, 3 wavelengths, 4 wavelengths, 5 wavelengths, 6 wavelengths, 7 wavelengths, 8 wavelengths, 9 wavelengths, 10 wavelengths, or any other number of wavelengths known in the art. In some embodiments, each wavelength within the first set of wavelengths may independently be from about 650 nm to about 950 nm. Each wavelength may be, for example, about 650 nm, about 655 nm, about 660 nm, about 665 nm, about 670 nm, about 675 nm, about 680 nm, about 685 nm, about 690 nm, about 695 nm, about 700 nm, about 705 nm, about 710 nm, about 715 nm, about 720 nm, about 725 nm, about 730 nm, about 735 nm, about 740 nm, about 745 nm, about 750 nm, about 755 nm, about 760 nm, about 765 nm, about 770 nm, about 775 nm, about 780 nm, about 785 nm, about 790 nm, about 795 nm, about 800 nm, about 805 nm, about 810 nm, about 815 nm, about 820 nm, about 825 nm, about 830 nm, about 835 nm, about 840 nm, about 845 nm, about 850 nm, about 855 nm, about 860 nm, about 865 nm, about 870 nm, about 875 nm, about 880 nm, about 885 nm, about 890 nm, about 895 nm, about 900 nm, about 905 nm, about 910 nm, about 915 nm, about 920 nm, about 925 nm, about 930 nm, about 935 nm, about 940 nm, about 945 nm, about 950 nm, or any range between any two of these values, including endpoints. In some embodiments, each wavelength within the first set of wavelengths may be greater than about 805 nm. In some embodiments, the average of the first set of wavelengths may be greater than about 805 nm. In certain embodiments, the first set of wavelengths may include five individual wavelengths to interrogate the targeted tissue: one in the red region below 730 nm, one in the NIR region below the 805 nm isosbestic point, one near or at the 805 nm isosbestic point, and two in the NIR region above the isosbestic point.

Turning to FIGS. 3 and 4, system 400 may include one or more electronics modules 302, e.g., 302A and 302B. Each electronics module 302A, 302B may be configured with the same or similar components; however, in some embodiments, electronics module 302A and 302B may be configured to detect and process different types of parameters of a user. For example, electronics module 302A may be configured to detect and process optical biometric properties of a user, while electronics module 302B may be configured to detect and process non-optical biometric properties of a user. In some embodiments, system 400 may include a single electronics module 302 that may include the hardware and functionality of electronics modules 302A and 302B, as discussed herein, and may be configured to carry out the program instructions for both electronics modules 302A and 302B, as discussed herein, within a single, integrated package.

In some embodiments, electronics module 302A may be communicatively coupled to the substrate 102, and may include a processor 310, a memory device 360, and instructions stored on the memory device 360 that may direct the processor 310 to perform one or more actions. As used herein, the term “communicatively coupled” means configured to communicate and/or connect via one or more methods of communication, such as a wireless connection (e.g., WiFi®), a wired connection, a Bluetooth connection, a near-field communication (NFC) connection, a radio frequency identification (RFID) connection, etc. The electronics module 302A may be configured to receive a signal from the one or more first detectors 104 mounted on the substrate 102, and process the received signal based on one or more first predefined algorithms. Based on the processed signal, the electronics module 302A may be configured to determine whether the user, e.g., on or to which substrate 102 is coupled, requires one or more first feedback actions.

In some embodiments, electronics module 302B may also be communicatively coupled to the substrate 102 and may include the same or similar components to that of electronics module 302A. Electronics module 302B may, however, be configured to receive a signal from the one or more second detectors 106 mounted on the substrate 102, and process the received signal based on one or more second predefined algorithms. Based on the processed signal, the electronics module 302B may be configured to determine whether the user, e.g., on or to which substrate 102 is coupled, requires one or more second feedback actions.

In some embodiments, at least one of the electronics modules 302A, 302B may be configured to determine that the user either requires no feedback actions, requires at least one of the first feedback actions, or requires at least one of the second feedback actions.

In some embodiments, the first and/or second feedback actions may include having an additional optical biometric property reading taken, having an additional non-optical biometric property reading taken, initiating one or more types of stimulation to be communicated to the user, and/or communicating with an external processor to enact a change in system behavior (e.g., biometrics of interest, operating mode, conditions of user stimulation, etc.).

In some embodiments, the first and/or second feedback actions may be provided to the user through changes in the user's real environment or virtual environment, e.g., via a VR device. In some embodiments, the first and/or second feedback actions may be perceived by the user, not perceived by the user, or partially perceived by the user. In some embodiments, the first and/or second feedback actions may be provided to the user pharmacologically, e.g., through intravascular, intramuscular, intraosseous, and/or other routes. In some embodiments, the first and/or second feedback actions may be provided to the user via some type of external environmental property, such as through a change in temperature, pressure, chemical composition, sound (e.g., continuous sound waves with changing pitch), light, motion, smell, feel, taste, and the like.

In some embodiments, the types of stimulation may be provided to the user via one or more terminals 110 mounted on the substrate 102 (FIGS. 1C-1D). In some embodiments, the types of stimulation may include tDCS, tACS, nVNS, TENS, TMS, ultrasound stimulation, optical stimulation, mechanical stimulation, or other types of invasive or non-invasive stimulation.

As shown in FIG. 3, the electronics module 302 may further include one or more environmental sensors 320, a communication interface 330, an I/O device 340, and an energy storage device 350 (e.g., a battery) configured to power the electronics module 302. Memory device 360 may include an operating system (OS) 370 and program 380, and a database 390. Operating system 370 may be a real-time operating system (RTOS) or program instructions in system firmware operating on the processor 310.

Program 380 may include stored instructions that direct the processor 310 to perform one or more steps toward processing signals received from substrate 102, as discussed above.

In some embodiments, the processor 310 may be configured to select the emitted set(s) of wavelengths and the respective distance(s) of the light source 108 from the first detectors 104, as discussed above. The input parameter may include, for example, a temperature, a lighting condition, a velocity, an acceleration, a change in acceleration, a pressure, a change in pressure, a volume, a change in volume, a measurement made, recorded, or calculated by the system, a communication from another device or system, or a combination thereof.

In some embodiments, the environmental sensor(s) 320 can measure parameters surrounding the patient and not the patient directly. Environmental properties may include, for example, temperature, humidity, pressure, motion, chemical composition, ambient light intensity, sound, etc., of the external environment in which the patient is positioned. For example, if the patient's body temperature is calculated as being too high, such as above some predetermined threshold, the electronics module 302, e.g., via the processor 310, may be configured to adjust a thermostat located in the room in which the patient is located. As another example, environmental sensor(s) 320 may include a microphone configured to receive spoken instructions informing the electronics module 302 how to operate.

In some embodiments, the electronics module 302 (e.g., 302A and/or 302B) may be configured to detect a configuration (e.g., a system architecture) of the substrate 102 when the electronics module 302 is communicatively connected to the substrate 102. Upon detecting the configuration of the substrate 102, electronics module 302 may be configured to adapt its own behavior to conform with the detected configuration. In some embodiments, electronics module 302 may include its own software and/or firmware, and upon being connected to the substrate 102, may be configured to determine whether the substrate 102 (e.g., with respect to its configuration) is compatible with the software and/or firmware. In some embodiments, if electronics module 302 determines that the substrate 102 is not compatible with the software and/or firmware, electronics module 302 may be configured to transmit an alert (e.g., to a computing device), and/or perform an update to its software and/or firmware such that the substrate 102 is compatible with the updated software and/or firmware. Electronics module 302 may be configured to update its software and/or firmware independently, e.g., via communicating internally with memory device 360, or dependently, e.g., via communicating with an external computing device to conduct an over-the-air (OTA) update.

In some embodiments, system 400 (FIG. 4) may include one or more components discussed herein, such as flexible material 202, substrate 102, one or more electronics modules 302 (e.g., 302A and/or 302B). In some embodiments, one or more of the components of system 400 may communicate via a network 402 such that, for example, electronics module 302 (e.g., 302A and/or 302B) may receive and process signals transmitted from substrate 102, as discussed herein.

In some embodiments, system 400 may be used to monitor multiple patients at one time (e.g., users who may or may not be geographically collocated). In some embodiments, the system may be configured to, for example, adjust a feedback device or environmental property, based on the different calculated biometric parameters across patients. In some embodiments, the biometric data calculated by the electronics module 302 (e.g., 302A and/or 302B) may be benefited by a patient other than the patient being monitored, for example, a patient who may be remote from the system.

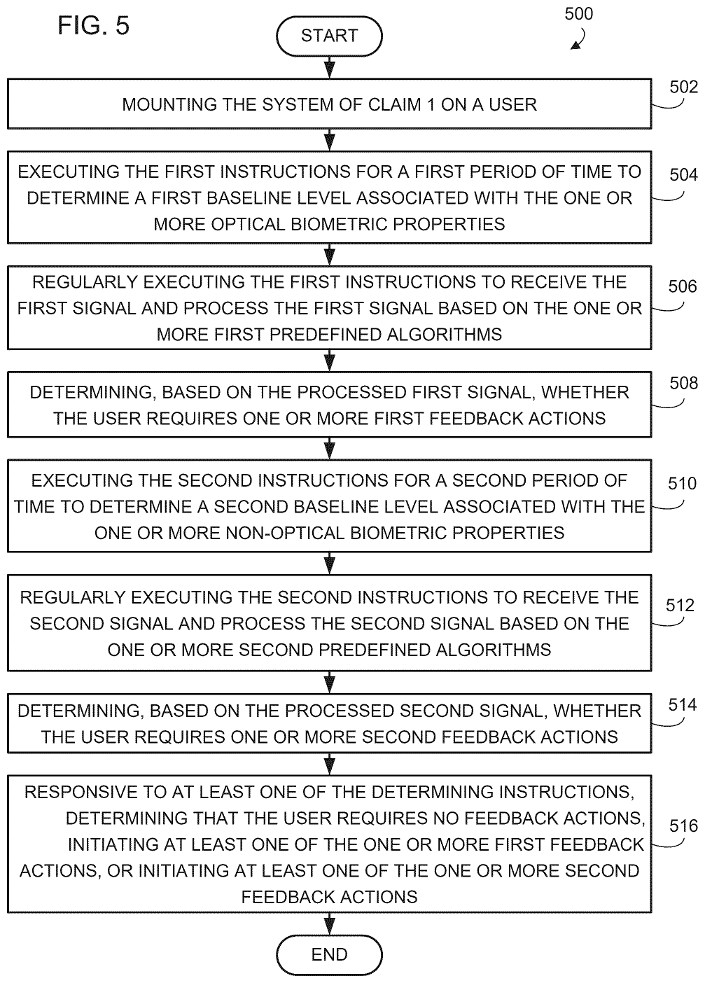

FIG. 5 is a flowchart of a method for detecting one or more biometric and neurophysiological parameters, in accordance with the present disclosure.

In block 502, the system 400 may be mounted on a user (e.g., on a part of the user's body, a piece of equipment and/or clothing worn by the user, etc.).

In block 504, the first instructions stored in the first memory device 360 of the electronics module 302A may be executed for a period of time (e.g., 2 minutes) to determine a baseline level associated with the one or more optical biometric parameters for the user.

In block 506, the first instructions may be regularly executed to receive the first signal and to process the first signal based on the one or more first predefined algorithms, as discussed herein.

In block 508, the first instructions may be further executed to determine, based on the processed first signal, whether the user requires one or more first feedback actions, as discussed herein.

In block 510, the second instructions stored in the second memory device 360 of the electronics module 302B may be executed for a period of time (e.g., 2 minutes) to determine a baseline level associated with the one or more non-optical biometric parameters for the user.

In block 512, the second instructions may be regularly executed to receive the second signal and to process the second signal based on the one or more second predefined algorithms, as discussed herein.

In block 514, the second instructions may be further executed to determine, based on the processed second signal, whether the user requires one or more second feedback actions, as discussed herein.

In block 516, responsive to at least one of the determining instructions, at least one of the first and second processors of the two respective electronics modules 302A, 302B may determine that the user requires no feedback actions, initiate at least one of the one or more first feedback actions, or initiate at least one of the one or more second feedback actions.

In some embodiments, the system 400 may integrate the functionality of electronics modules 302A and 302B into one electronics module 302 assimilating the hardware, functionality, and carrying out of the program instructions for both electronics modules within a single package.

In some embodiments, the system 400 may further include an external computing device including a memory and a computer processor. The external computing device may be connected to at least a portion of at least one of the processor and the memory device via a connection, wherein at least a portion of the program instructions is also stored on the external computing device.

The substrate 102 and/or the electronics module 302 can include a communication or connection interface 330. In some embodiments, the communication interface 330 can facilitate connections that can be, for example, a wireless connection (e.g., WiFi®), a wired connection, a Bluetooth connection, an NFC connection, a RFID connection, or a combination thereof. In some embodiments, data processing and real-time feedback may occur within the components onboard the substrate, or offboard through communication with the external computing device. The external computing device may comprise, for example, a smartphone, a charging or communications base station, a display screen, a tablet, a computer, a mobile or web-based application, or another device.

In some embodiments, the systems disclosed herein can be networked for concurrent monitoring of different physiological conditions of a user, the same or different physiological conditions at different locations on the body of a user, one or more physiological conditions of a group of wearers in a population, or a combination thereof.

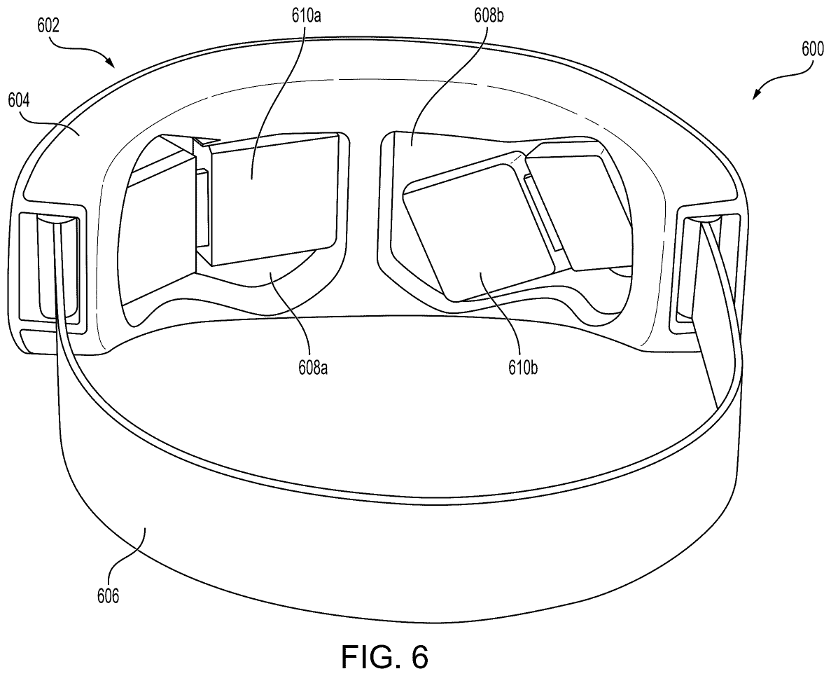

FIG. 6 depicts a system 600 that can be used for detecting biometric and/or neurophysiological parameters of a user. The system 600 can include a wearable device 602, such as a visor or headset, that can be worn on the user's body, such as on the user's head (e.g., forehead). The wearable device 602 can include a first portion 604 configured to fit across a user's forehead, and an adjustable strap 606 used to tighten or loosen the wearable device 602 such that it fits properly on the user's head. The wearable device 602 can also include one or more cutouts or slots 608 (e.g., 608a, 608b) configured to house one or more components of the system 600, such as one or more substrates 610 (e.g., 610a, 610b). In such embodiments, the substrate(s) 610 may include one or more of the same or similar features as substrate 102, as discussed above. However, substrate(s) 610 may be configured such that it can fit and/or lock into the slot(s) 608. In this way, substrate(s) 610 can aid in the system 600 being a modular system. For example, a first substrate 610a (e.g., monitoring one or more optical biometric properties of the user) may be integrated into the wearable device 602 (e.g., via slot 608a), while a second substrate 610b (e.g., monitoring one or more non-optical biometric properties of the user) may be attached to another part of the user's body, such as the user's chest. In this way, the system 600 can be configured to monitor or detect biometric and/or neurophysiological parameters of the user from one or more of the user's body parts.

System 600 can also include one or more electronics modules 302, as discussed above, that can themselves be integrated into the wearable device 602 and/or be disposed external to the wearable device 602 (e.g., via a wired or wireless connection). The electronics module(s) 302 of the system 600 can also be configured in a modular fashion including, for example, one or more electronics modules 302 integrated into the wearable device 602 (e.g., internal to a substrate 610, or external to the substrate 610 but still integrated into the wearable device 602 via another slot 608), and/or one or more electronics modules 302 that are external to the wearable device 602 (e.g., via a wired or wireless connection).

System 600 can also include one or more components to support the wearable device 602 being a remote or wireless device. For example, wearable device 602 can include a rechargeable battery. In some embodiments, the wearable device 602 may include one or more internal processors that may be integrated into the wearable device 602 but are separate from the electronics module(s) 302. These internal processor(s) may include one or more of the same or similar features as electronics module(s) 302, as discussed herein.

Although some of the processing systems described herein can be embodied in software or code executed by general purpose hardware as discussed above, as an alternative the same can also be embodied in dedicated hardware or a combination of software/general purpose hardware and dedicated hardware. If embodied in dedicated hardware, each can be implemented as a circuit or state machine that employs any one of or a combination of a number of technologies. These technologies can include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc.

In some embodiments, the systems and methods described herein include independent wireless devices communicating biometrics information about different areas of tissue (e.g., the brain) simultaneously. In some embodiments, the systems and methods described herein include scanning a single device over different areas of the body and continuously imaging tissue, changing methods based on determined tissue state or changes in patient condition.

In some embodiments, the systems and methods described herein include two or more independent systems that can simultaneously interrogate multiple areas of cerebral and somatic tissue to interrelate physiological status (for example, tissue oxygenation) in each area. These areas may have significantly different oxygenation signatures at any given time and simultaneously sampling these is particularly important to understand situations of local or central fatigue or recovery onset by the user. Simultaneous imaging of different body systems can also elucidate generalized physiological condition, for instance indicating systemic response to exogenous conditions such as carbon monoxide poisoning or endogenous conditions such as hemorrhage. The independently sampled processed data from each area of the body may then send signals to a user interface if a specific tissue level, condition, or status is reached, or stream data to the external processing module for real-time interpretation, or both.

In some embodiments, the functional near-infrared spectroscopy systems and methods described herein include independent wireless devices communicating multi-point physiological information (e.g., oxygenation) about the brain and body simultaneously.

In some embodiments, the systems and methods described herein include multiple systems that can be worn by multiple different individuals whose data is integrated to form a comprehensive image of a group of individuals' health. This integration can be simultaneous for co-located users or asynchronous for disparate groups, or another combination. For example, comparing real-time physiological monitoring across multiple individuals can enable population monitoring and a more holistic image of group performance and wellness. Such continuous imaging can identify early threats or enhancements and increase risk or opportunity for better group performance and outcome.

As an example, a set of n systems 400 may be placed on the heads or bodies of n users. Each system is as described herein and includes an LED user interface light indicating a green/yellow/red indication of tissue health. Based on individual physiology of the n users, the n systems 400 monitor users in this cohort depending on the individual user's needs. In an example, one system in the user cohort begins sending abnormal signals back to the external processing unit indicating the onset of cognitive or physiological change in the target user that may have implications for the state of the rest of the user cohort, providing earlier notification from earlier surveillance and allowing real-time adaptation to monitored changes in condition.

In some embodiments, the systems and methods described herein include monitoring population health through a network of individual users' biometric detection systems. In some embodiments, this can enable broader decision making and earlier insight into performance degradations or risks from proximity to decompensating near neighbors. For example, the monitored conditions can include pre-symptomatic detection of infection, fatigue, or environmental exposure and the implementation of remedial strategies to optimize outcomes.

Where any component discussed herein is implemented in the form of firmware and/or software, any one of a number of programming languages may be employed such as, for example, C, C++, C#, Objective C, Java®, JavaScript®, Perl, PHP, Visual Basic®, Python®, Ruby, Flash®, or other programming languages. A number of firmware and/or software components are stored in the memory and are executable by the processor. In this respect, the term “executable” means a program file that is in a form that can ultimately be run by the processor. Examples of executable programs can be, for example, a compiled program that can be translated into machine code in a format that can be loaded into a random access portion of the memory and run by the processor, source code that can be expressed in proper format such as object code that is capable of being loaded into a random access portion of the memory and executed by the processor, or source code that can be interpreted by another executable program to generate instructions in a random access portion of the memory to be executed by the processor, etc. An executable program can be stored in any portion or component of the memory including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

The memory is defined herein as including both volatile and nonvolatile memory and data storage components. Volatile components are those that do not retain data values upon loss of power. Nonvolatile components are those that retain data upon a loss of power. Thus, the memory can include, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM can include, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor can represent multiple processors and/or multiple processor cores and the memory can represent multiple memories that operate in parallel processing circuits, respectively. In such a case, the local interface can be an appropriate network that facilitates communication between any two of the multiple processors, between any processor and any of the memories, or between any two of the memories, etc. The local interface can include additional systems designed to coordinate this communication, including, for example, performing load balancing. The processor can be of electrical or of some other available construction.

Although some of the processing systems described herein can be embodied in software or code executed by general purpose hardware as discussed above, as an alternative the same can also be embodied in dedicated hardware or a combination of software/general purpose hardware and dedicated hardware. If embodied in dedicated hardware, each can be implemented as a circuit or state machine that employs any one of or a combination of a number of technologies. These technologies can include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc.

It should be understood that any logic or application described herein that incorporates software or code can be embodied in any non-transitory computer-readable medium for use by or in connection with an instruction execution system such as, for example, a processor in a computer system or other system. In this sense, the logic can include, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system. The computer-readable medium can incorporate any one of many physical media such as, for example, magnetic, optical, or semiconductor media. More specific examples of a suitable computer-readable medium include, but are not limited to, magnetic tapes, magnetic floppy diskettes, magnetic hard drives, memory cards, solid-state drives, USB flash drives, or optical discs. Also, the computer-readable medium can be a random access memory (RAM) including, for example, static random access memory (SRAM) and dynamic random access memory (DRAM), or magnetic random access memory (MRAM). In addition, the computer-readable medium can be a read-only memory (ROM), a programmable read-only memory (PROM), an erasable programmable read-only memory (EPROM), an electrically erasable programmable read-only memory (EEPROM), or other type of memory device.

Further, any logic or application described herein can be implemented and structured in a variety of ways. For example, one or more applications described can be implemented as modules or components of a single application. Further, one or more applications described herein can be executed in shared or separate computing devices or a combination thereof. For example, a plurality of the applications described herein can execute in the same computing device 515, or in multiple computing devices in the same computing environment. Additionally, it is understood that terms such as “application,” “service,” “system,” “engine,” “module,” and so on may be interchangeable and are not intended to be limiting.

While various illustrative embodiments incorporating the principles of the present teachings have been disclosed, the present teachings are not limited to the disclosed embodiments. Instead, this application is intended to cover any variations, uses, or adaptations of the present teachings and use its general principles. Further, this application is intended to cover such departures from the present disclosure that are within known or customary practice in the art to which these teachings pertain.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

What is claimed is:

1. A system comprising:
a substrate in contact with a user and comprising:
one or more first detectors capable of detecting one or more optical biometric properties of the user; and
one or more second detectors capable of detecting one or more non-optical biometric properties of the user;
a first electronics module communicatively coupled to the substrate and comprising:
a first processor;
a first memory device; and
first instructions stored on the first memory device that, when executed, direct the first processor to:
receive a first signal from the one or more first detectors;
process the first signal based on one or more first predefined algorithms; and
determine, based on the processed first signal, whether the user requires one or more first feedback actions; and
a second electronics module communicatively coupled to the substrate and comprising:
a second processor;
a second memory device; and
second instructions stored on the second memory device that, when executed, direct the second processor to:
receive a second signal from the one or more second detectors;
process the second signal based on one or more second predefined algorithms; and
determine, based on the processed second signal, whether the user requires one or more second feedback actions;
wherein at least one of the first processor and the second processor:
responsive to at least one of the determining instructions,
determine that the user requires no feedback actions,
initiate at least one of the one or more first feedback actions, or
initiate at least one of the one or more second feedback actions.

2. The system of claim 1, wherein:
the substrate further comprises a first light source capable of emitting a first set of wavelengths of red or near-infrared light;
the one or more first detectors are capable of detecting the first set of wavelengths and are mounted on the substrate at a first distance from the first light source; and
the first instructions further direct the first processor to:
detect a physical configuration of the substrate;
select, based on the physical configuration, the first set of wavelengths; and
select, based on the physical configuration, the first distance from the first light source,
wherein receiving the first signal is based on the physical configuration.

3. The system of claim 1, wherein the one or more optical biometric properties comprise one or more of tissue oxygenation, heart rate, respiratory rate, oxygen saturation, blood pressure, or combinations thereof.

4. The system of claim 1, wherein the one or more non-optical biometric properties comprise one or more of electroencephalography (EEG), electrooculography (EOG), electromyography (EMG), bioimpedance circuitry, thermal properties, mechanical properties, or combinations thereof.

5. The system of claim 1, wherein the one or more first and second feedback actions comprise one or more of an optical biometric property reading, a non-optical biometric property reading, one or more types of stimulation, or combinations thereof.

6. The system of claim 5, wherein:
the substrate further comprises one or more terminals capable of providing a user with the one or more types of stimulation; and
the one or more types of stimulation comprise one or more of transcranial direct current stimulation (tDCS), transcranial alternating current stimulation (tACS), vagus nerve stimulation (nVNS), transcutaneous electrical nerve stimulation (TENS), transcranial magnetic stimulation (TMS), ultrasound stimulation, optical stimulation, mechanical stimulation, or combinations thereof.

7. The system of claim 1, wherein the one or more first and second feedback actions are provided to the user by changes in a real or virtual environment of the user, and/or by pharmacological changes.

8. The system of claim 7, wherein the one or more first and second feedback actions are perceived by the user, are not perceived by the user, or are partially perceived by the user.

9. The system of claim 1, wherein the one or more first and second feedback actions are provided to a user via an environmental property comprising one or more of temperature, pressure, chemical composition, sound, light, motion, or combinations thereof.

10. The system of claim 1, further comprising a flexible material coupled to the substrate to improve contact with the user.

11. The system of claim 10, wherein the flexible material and the substrate are configured to removably contact one or more of a body part of the user, an article of clothing, a piece of equipment, a prosthetic, or combinations thereof.

12. A system comprising:
a substrate in contact with a user and comprising:
one or more first detectors capable of detecting one or more optical biometric properties of the user;
one or more second detectors capable of detecting one or more non-optical biometric properties of the user; and
one or more terminals capable of providing the user with one or more types of stimulation;
a first electronics module communicatively coupled to the substrate and comprising:
a first processor;
a first memory device; and
first instructions stored on the first memory device that, when executed, direct the first processor to:
receive a first signal from the one or more first detectors;
process the first signal based on one or more first predefined algorithms; and
determine, based on the processed first signal, whether the user requires one or more first types of stimulation; and
a second electronics module communicatively coupled to the substrate and comprising:
a second processor;
a second memory device; and
second instructions stored on the second memory device that, when executed, direct the second processor to:
receive a second signal from the one or more second detectors;
process the second signal based on one or more second predefined algorithms; and
determine, based on the processed second signal, whether the user requires one or more second types of stimulation;
wherein at least one of the first processor and the second processor:
responsive to at least one of the determining instructions,
determine that the user requires no types of stimulation,
initiate, via the one or more terminals, at least one of the one or more first types of stimulation, or
initiate, via the one or more terminals, at least one of the one or more second types of stimulation.

13. The system of claim 12, wherein:
the substrate further comprises a first light source capable of emitting a first set of wavelengths of red or near-infrared light;
the one or more first detectors are capable of detecting the first set of wavelengths and are mounted on the substrate at a first distance from the first light source; and
the first instructions further direct the first processor to:
detect a physical configuration of the substrate;
select, based on the physical configuration, the first set of wavelengths; and
select, based on the physical configuration, the first distance from the first light source,
wherein receiving the first signal is based on the physical configuration.

14. The system of claim 12, wherein the one or more optical biometric properties comprise one or more of tissue oxygenation, heart rate, respiratory rate, oxygen saturation, blood pressure, or combinations thereof.

15. The system of claim 12, wherein the one or more non-optical biometric properties comprise one or more of electroencephalography (EEG), electrooculography (EOG), electromyography (EMG), bioimpedance circuitry, thermal properties, mechanical properties, or combinations thereof.

16. The system of claim 12, wherein the one or more first and second types of stimulation comprise one or more of transcranial direct current stimulation (tDCS), transcranial alternating current stimulation (tACS), vagus nerve stimulation (nVNS), transcutaneous electrical nerve stimulation (TENS), transcranial magnetic stimulation (TMS), ultrasound stimulation, optical stimulation, mechanical stimulation, or combinations thereof.

17. The system of claim 12, further comprising a flexible material coupled to the substrate to improve contact with the user, wherein the flexible material and the substrate are configured to removably contact one or more of a body part of the user, an article of clothing, a piece of equipment, a prosthetic, or combinations thereof.

18. A system comprising:
a substrate in contact with a user and comprising:
one or more first detectors capable of detecting one or more optical biometric properties of the user; and
one or more second detectors capable of detecting one or more non-optical biometric properties of the user; and
an electronics module communicatively coupled to the substrate and comprising:
one or more processors;
one or more memory devices; and
one or more instructions stored on the one or more memory devices that, when executed, direct the one or more processors to:
receive a first signal from the one or more first detectors;
receive a second signal from the one or more second detectors;
process the first signal based on one or more first predefined algorithms;
process the second signal based on one or more second predefined algorithms;
determine, based on the processed first signal, whether the user requires one or more first feedback actions; and
determine, based on the processed second signal, whether the user requires one or more second feedback actions;
wherein at least one of the one or more processors:
responsive to at least one of the determining instructions,
determine that the user requires no feedback actions,
initiate at least one of the one or more first feedback actions, or
initiate at least one of the one or more second feedback actions.

19. The system of claim 18, wherein:
the substrate further comprises one or more terminals capable of providing a user with one or more types of stimulation; and
the one or more types of stimulation comprise one or more of transcranial direct current stimulation (tDCS), transcranial alternating current stimulation (tACS), vagus nerve stimulation (nVNS), transcutaneous electrical nerve stimulation (TENS), transcranial magnetic stimulation (TMS), ultrasound stimulation, optical stimulation, mechanical stimulation, or combinations thereof.

20. A method of calculating one or more biometric properties, the method comprising:
mounting the system of claim 1 on a user;
executing the first instructions for a first period of time to determine a first baseline level associated with the one or more optical biometric properties;
regularly executing the first instructions to process the first signal based on the one or more first predefined algorithms;
determining, based on the processed first signal, whether a user requires one or more first feedback actions;
executing the second instructions for a second period of time to determine a second baseline level associated with the one or more non-optical biometric properties;
regularly executing the second instructions to process the second signal based on the one or more second predefined algorithms; and
determining, based on the processed second signal, whether the user requires one or more second feedback actions;
wherein at least one of the first processor and the second processor:
responsive to at least one of the determining instructions,
determining that the user requires no feedback actions,
initiating at least one of the one or more first feedback actions, or
initiating at least one of the one or more second feedback actions.
